(19) United States
(12) Patent Application Publication
(10) Pub. No.: US 2017/0332994 A1
(43) Pub. Date: Nov. 23, 2017

(54) DEVICE FOR AUSCULTATION

(71) Applicants: Jason Robke, Avon, OH (US); Jennifer Robke, Avon, OH (US)

(72) Inventors: Jason Robke, Avon, OH (US); Jennifer Robke, Avon, OH (US)

(21) Appl. No.: 15/158,022

(22) Filed: May 18, 2016

(51) Int. Cl. A61B 7/04; A61B 5/00; A61B 5/024; G09B 23/28

(52) U.S. Cl. CPC A61B 7/04; G09B 23/28; A61B 5/7415; A61B 5/742; A61B 5/0002; A61B 5/6898; A61B 5/024; A61B 2560/0475

(57) ABSTRACT

A device for auscultation of sounds generated by a patient having a stethoscope head operatively connected to a sound transference means and a smartphone operatively connected to the sound transference means, wherein the smartphone stores the sounds generated by a patient and displays a graphic representation of the sounds generated by the patient.

### BACKGROUND OF THE INVENTION

Clinical use of stethoscope is important in diagnostic medicine. Sounds generated by the heart are useful in diagnosis and treatment of heart diseases. Sounds from the lungs and bowels and other anatomical structures are also important in medical treatment. Traditionally, an acoustic stethoscope has been used to hear, or auscultate, sounds from patients' anatomical structures and organs.

Traditional uses of the acoustic stethoscope includes limitations. While a clinician can later record what is heard through the stethoscope, there is no record of the actual sounds generated by the patient's body. This can limit communication about the patient's medical condition, including the ability to teach and train healthcare professional. There remains a long-felt need for a suitable means of improving auscultation in the medical field.

### BRIEF DESCRIPTION OF THE DRAWINGS

FIG. 1 shows a device for auscultation in accordance with the present invention.

FIG. 2 shows a top view of human chest anatomy showing placement of the device of FIG. 1 for auscultation in accordance with the present invention.

FIG. 3 shows a back view and side view of human anatomy showing placement of the device of FIG. 1 for pulmonary auscultation in accordance with the present invention.

FIG. 4 shows a front view of human anatomy showing placement of the device of FIG. 1 for abdominal auscultation in accordance with the present invention.

FIG. 5 shows an alternative embodiment of a device for auscultation in accordance with the present invention.

### DETAILED DESCRIPTION OF THE INVENTION

Preliminarily, it should be noted that certain terms used herein, such as for example above, below, upper, lower left and right, are used to facilitate the description of the invention. Unless otherwise specified or made apparent by the context of the discussion, such terms and other directional terms should be interpreted with reference to the figure(s) under discussion. Such terms are not intended as a limitation on the position in which the invention or components may be used. Indeed, it is contemplated that the components of the invention may be easily positioned in any desired orientation for use. Likewise, numerical terms such as for example “first”, and “second” are not intended as a limitation or to imply a sequence, unless otherwise specified or made apparent by the context of the discussion. The term “operatively connected” is understood to include a linking together of the portions under consideration and may include a physical engagement and/or a functional or operational connection.

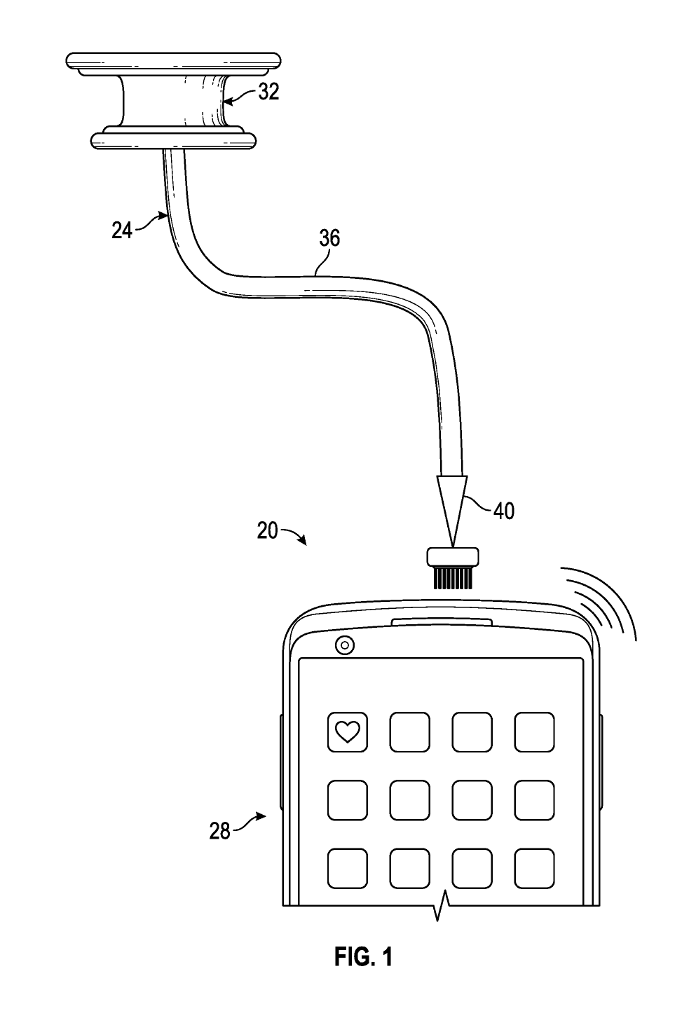

Referring now to the drawings, there is illustrated in FIG. 1 a device for improving auscultation of a patient indicated generally at 20, according to the invention. The term “auscultation” may be understood to include listening to sounds arising within or around organs (such as the heart or lungs) or other anatomical structures as an aid to diagnosis and treatment.

The device 20 shown includes a sound transference means 24 and a processor 28. The sound transference means 24 shown includes a tube 36 secured to a head 32. The tube 36 shown is generally hollow and flexible to allow ready positioning of the head 32 as desired by the user. The head 32 shown is a stethoscope head, a drum-like structure which includes a diaphragm which operates to receive and transmit physical vibration and sound waves. The head 32 may be constructed of metal or other suitable material and may be double-sided for use with patients of differing sizes and different areas of the body. The head 32 may have deep cups that capture sounds from the target area and may be ringed with a “chill ring” to keep the patient from being uncomfortable when touched by a cold metal head.

The tube 36 may be any suitable length, including about forty-six inches. The tube 36 may be constructed of any suitable material, including non-latex based materials adapted for patients sensitive to latex. The illustrated sound transference means 24 includes a coupling 40 operatively connected to the tube 36. The coupling 40 may vary in size and construction to operatively connect the head 32 and the tube 36 to a processor 28.

The processor 28 may be any suitable smartphone, notebook-style computer, portable computer, or the like. The device 20 may employ any computer or other apparatus adapted to receive and process information from the head 32. The processor 28 may employ components to carry out instructions of a computer program. The processor 28 may also employ an application to process signals detected by the head 32. The processor 28 may likewise store, retrieve and transmit information, including sound, as desired by the user.

The device 20 may operate without the tube 36. In such a configuration, the device 20 employs a head 32 adapted to transmit a signal to the processor 28 by means of ambient air and a wireless mechanism, such as radio waves instead of wires or cables to operatively connect the head 32 and the processor 28.

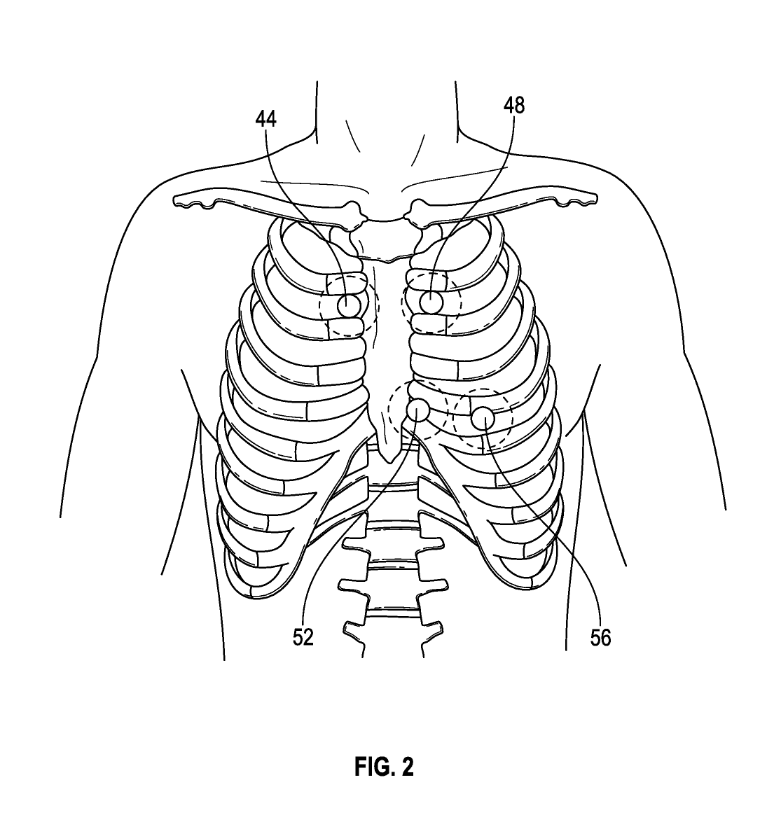

In operation, the head 32 of the device 20 may be placed on a patient as desired by a healthcare provider such as a nurse, veterinarian, physician, pharmacist, and the like. Referring now to FIG. 2, known anatomical locations may be employed on the human chest region for auscultation in the diagnosis and treatment of disease. Shown are four common anatomical locations used by clinicians in auscultation of a patient—the aortic area 44, the pulmonic area 48, the tricuspid area 52, and the mitral area 56. Similarly, the back of the patient may be auscultated in an attempt to hear sounds generated by the pulmonary system. The abdomen may also be auscultated for bowel sounds and bruits in the renal arteries.

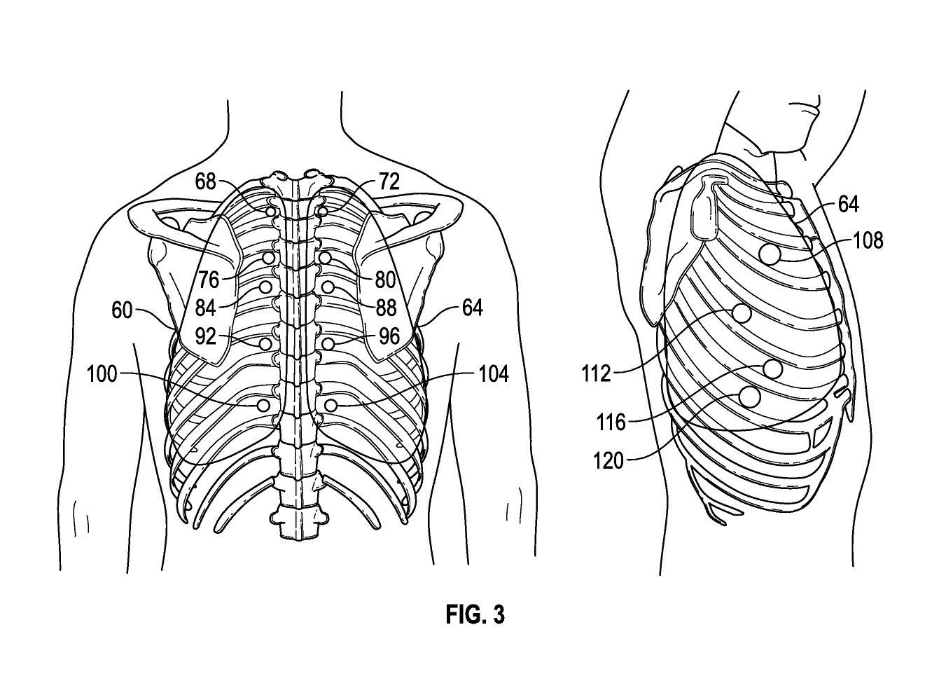

Referring now primarily to FIG. 3, the left lung 60 and the right lung 64 are shown in a typical depiction of human anatomy. The head 32 of the device 20 may be placed on the left lung 60 and right lung 64 as desired by the user. Pulmonary sounds may vary in duration and tone with various states of infirmity and placement of the head 32 of the device 20. The user of the device 20 learns, with experience and instruction, to associate certain sounds and changes in sound from the left lung 60 and the right lung 64 with pathology and changes in pulmonary function.

Referring still to FIG. 3, the left lung 60 presents for auscultation with the head 32 of the device 20 at locations 68, 76, 84, 92, and 100. The right lung 64 similarly presents for auscultation at locations 72, 80, 88, 96, and 104. No particular sequence in placement of the head 32 of the device 20 for auscultation should be inferred from the numerical sequence supplied in FIG. 3. The right lung 64 may similarly presents for auscultation at locations 108, 112, 116, 120—as viewed from the right side of the patient.

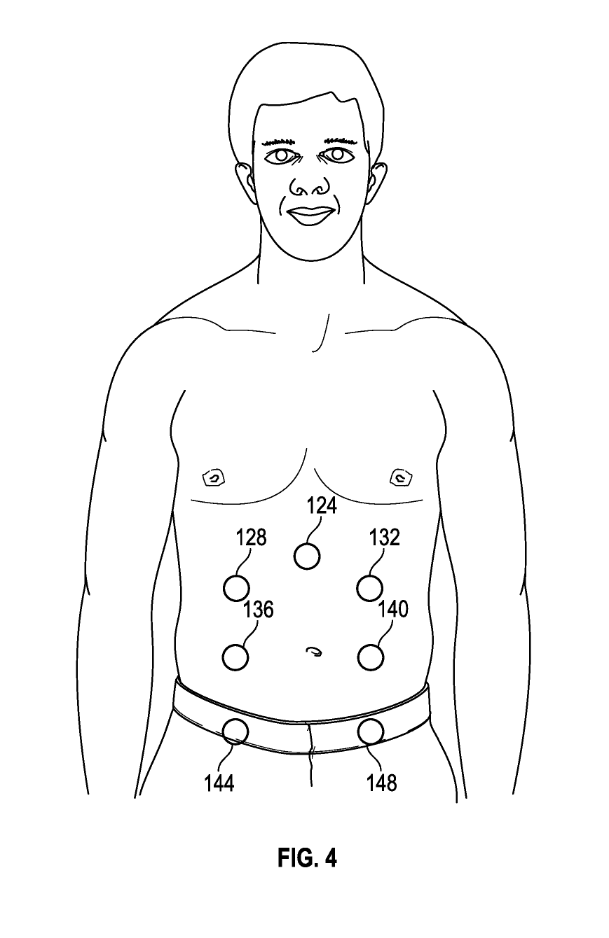

Referring now primarily to FIG. 4, the device 20 is also suitable for auscultation at selected anatomical locations of the abdomen. Bruits, or turbulent-like sounds, may be heart at the abdomen aorta, indicated generally at 124. The renal arteries 128, 132 may similarly provide sounds detected by the head 32 of the device 20. The iliac arteries 136, 140 and the femoral arteries 144, 148 likewise can provide valuable diagnostic sounds as can be detected by the head 32 of the device 20.

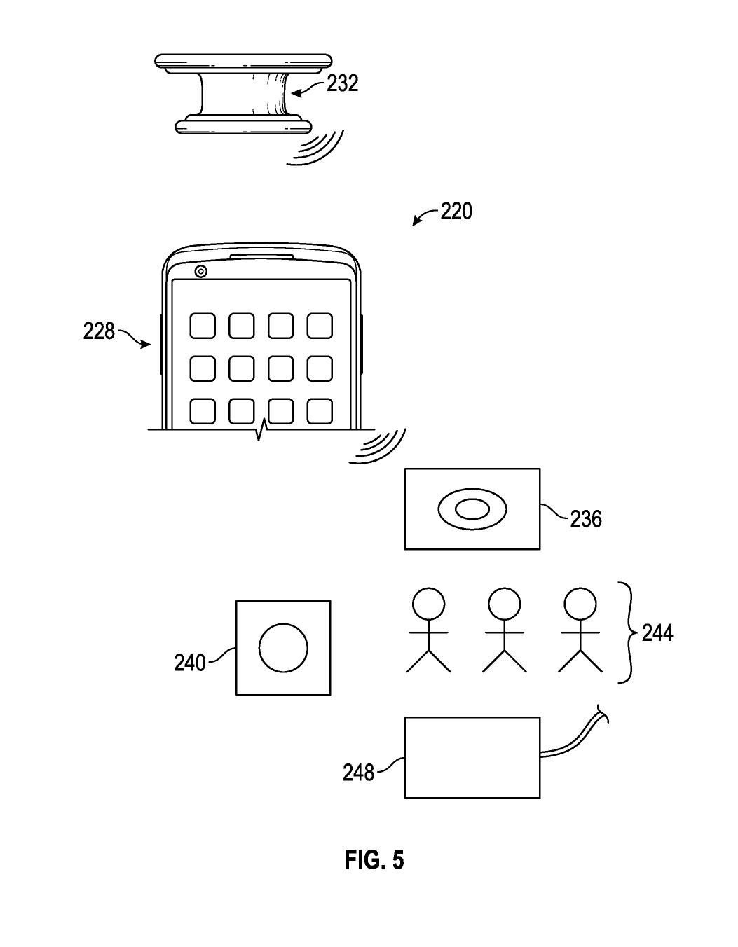

Referring now primarily to FIG. 5, an alternate embodiment of the device 220 is shown. A head 232 of the device 220 shown is a drum-like structure which includes a diaphragm which operates to receive and transmit physical vibration and sound waves. The head 232 may be constructed of metal and may be double-sided for use with patients of differing sizes and different areas of the body. The head 232 may have deep cups that capture sounds from the target area and may be ringed with a “chill ring” to keep the patient from being uncomfortable when touched by the cold metal head.

The head 232 is operatively connected to a processor 228, which may be any suitable smartphone, notebook-style computer, portable computer, or the like. The device 220 may employ any computer or other apparatus adapted to receive and process information from the head 232. The processor 228 may employ components to carry out instructions of a computer program. The processor 228 may also employ an application to process signals detected by the head 232. The processor 228 may likewise store, retrieve and transmit information, including sound, as desired by the user.

The head 232 may be operatively connected to the processor 228 by means of electromagnetic radiation. The head 232 may include a suitable power source, transmitter, and/or amplifier. Without limitation and for purposes of illustration, a Bluetooth, Wi-Fi or other suitable wireless local area networks (WLAN) or wireless personal area networks (WPAN) or the like may be used to operatively connect the head 232 to the processor 228.

The processor 228 may be operatively connected to a speaker 236 and/or graphic display 240 for presentation to an audience 244. The graphic display 240 may be a light emitting diode monitor, liquid crystal display monitor, tangible printed strip, or other suitable display means. The audience 244 may be any suitable number of persons, such as students, clinicians, and the like. The processor 28, 228 may be operatively connected to a storage device 248 for later viewing, retrieval and/or additional processing. The storage drive may be of any suitable sort including but not limited to hard drives, USB drives, solid state, removable storage, RAM, mass storage device, flash memory chips, or the like.

The device 20, 220 may be employed in a wide variety of settings for many purposes. Standard stethoscopes provide clinical information to only one listener, do not store the sounds generated by the patient, and do not provide for optimal teaching and training of students and newer clinicians. The device 20 may process the sounds from a patient's body, display graphic representations of the sounds, convert the sounds to electronic format, store the sounds, amplify the sounds, and transmit the sounds for remote storage, use by a listener, and other uses.

The device 20, 220 may employee one or more applications, also known as “apps,” in conjunction with the processor 228 to facilitate operation in the clinical or teaching setting. The applications may be used by the processor 28, 228 in conjunction with other components to process sounds generated by the patient's body. The applications may also be employed to generate auditory or graphic representations of the sounds generated by the patient's body.

The invention may be made from any suitable material and by any suitable method. The invention may be adapted to fit a wide variety of uses. It will be appreciated that the components of the invention may be modified as needed to accommodate varying sizes and shapes and applications.

It is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. The disclosure may readily be utilized as a basis for the designing of other structures, methods and systems for carrying out the present invention. It is important, therefore, that the claims be regarded as including equivalent constructions. Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract and disclosure are neither intended to define the invention of the application, which is measured by the claims, nor are they intended to be limiting as to the scope of the invention in any way.

What is claimed is:

1. A device for auscultation of sounds generated by a patient's body comprising:
a stethoscope head operatively connected to a sound transference means; and
a smartphone operatively connected to the sound transference means and the stethoscope head,
wherein the smartphone receives the sounds generated by the patient's body and provides a representation of the sounds generated by the patient's body.

2. The device of claim 1 further comprising a speaker, wherein the speaker amplifies a representation of the sounds generated by the patient's body and a listener is instructed with the representation of the sounds generated by the patient's body.

3. The device of claim 2 wherein the listener is a medical student.

4. The device of claim 1 wherein the representation of the sounds generated by the patient's body is an audible sound.

5. The device of claim 4 wherein the audible sound is used to instruct a student.

6. The device of claim 1 further comprising a storage device operatively connected to the smartphone.

7. The device of claim 1 further comprising a graphic display operatively connected to the smartphone.

8. The device of claim 7 further comprising a storage device operatively connected to the smartphone.

9. The device of claim 8 wherein the storage device is a solid state drive.

10. The device of claim 1 wherein the sounds generated by the patient's body include a heartbeat.

11. A device for wireless auscultation of sounds generated by a patient's body comprising:
a stethoscope head operatively connected to a power supply; and
a smartphone operatively connected to the stethoscope head,
wherein the smartphone receives the sounds generated by the patient's body and provides a representation of the sounds generated by the patient's body.

12. The device of claim 11 further comprising a speaker, wherein the speaker amplifies a representation of the sounds generated by the patient's body and a listener is instructed with the representation of the sounds generated by the patient's body.

13. The device of claim 12 wherein the listener is a medical student.

14. The device of claim 11 further comprising a storage device operatively connected to the smartphone.

15. A device for training in auscultation of sounds generated by a patient's body comprising:
a stethoscope head; and
a smartphone operatively connected to the stethoscope head,
wherein the smartphone receives from the stethoscope head the sounds generated by the patient's body and provides a representation of the sounds generated by the patient's body.

16. The device of claim 15 further comprising a power supply operatively connected to the stethoscope head.

17. The device of claim 16 further comprising a graphic display operatively connected to the smartphone.

18. The device of claim 15 further comprising a sound transference means operatively connected to the stethoscope head.

19. The device of claim 18 further comprising a graphic display operatively connected to the smartphone.

20. The device of claim 19 further comprising a storage device operatively connected to the smartphone.
